(19) United States
(12) Patent Application Publication
(10) Pub. No.: US 2009/0281473 A1
(43) Pub. Date: Nov. 12, 2009

(54) Anti-Human Immunodeficiency Virus Surrogate Target Agent Technology Filter Intended to Neutralize or Remove Human Immunodeficiency Virus Virions From Blood

(76) Inventors: Lane Bernard Scheiber, Annandale, VA (US); Lane Bernard Scheiber, II, Grosse Ile, MI (US)

Correspondence Address: LANE B. SCHEIBER, 7864 OAK RIVER DRIVE, GROSS ILE, MI, 48138, US

(21) Appl. No.: 12/116,942

(22) Filed: May 7, 2008

(51) Int. Cl. A61M 1/34

(52) U.S. Cl. 604/5.02; 604/6.09

(57) ABSTRACT

The Human Immunodeficiency Virus posses a significant threat to the world's population. Current strategies utilized to treat infectious agents have not been adequate to contain and eradicate this deadly viral infection. HIV seeks out its host, a T-Helper cell, by utilizing glycoprotein 120 probes to engage a CD4 cell-surface receptor located on the surface of a T-Helper cell. Developing blood filtering techniques that incorporate filter mediums that offer HIV virion's probes the opportunity to engage the cell-surface receptors they are seeking offers a means of neutralizing and removing HIV. Filtering the blood of a patient with filter mediums comprised of T-Helper cells, sheets of lipid bilayer or virus-like structures with each type of medium possessing cell-surface receptors intended to attract and engage HIV virions provides an effective strategy to prevent and treat AIDS.

### CROSS-REFERENCE TO RELATED APPLICATIONS

None.

### STATEMENT REGARDING SPONSORED RESEARCH OR DEVELOPEMNT

Not applicable.

### REFERENCE TO SEQUENCE LISTING, A TABLE, OR COMPUTER LISTING COMPACT DISC APPENDIX

Not applicable.

©2008 Lane B. Scheiber and Lane B. Scheiber II. A portion of the disclosure of this patent document contains material, which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND OF THE INVENTION

1. Field of the Invention

This invention relates to any medical device that is utilized to filter the blood of a patient infected with the Human Immunodeficiency Virus with the intention of neutralizing or removing from the blood infectious Human Immunodeficiency Virus virions.

2. Description of Background Art

It is estimated by the Center for Disease Control that in the United States 55,000 to 60,000 new cases of Human Immunodeficiency Virus (HIV) are occurring each year. It is thought that there are 900,000 people currently infected with HIV in the United States, with many victims not aware that they have contracted the virus. Further, it has been estimated that the Human Immunodeficiency Virus (HIV), the pathogen that causes Acquired Immune Deficiency Syndrome (AIDS), has infected as many as 30-60 million people around the globe.

The presence of HIV first came to the general attention of those in the United States in 1981, when there appeared an outbreak of Kaposi's Sarcoma and Pneumocystis carinii pneumonia in gay men in New York and California. After over twenty-five years of research and investigation, eradicating the ever growing global humanitarian crisis posed by the HIV remains an elusive goal for the medical community. It is estimated the virus has already killed 25 million citizens of this planet.

The Human Immunodeficiency Virus has been previously referred to as human T-Lymphotrophic virus III (HTLV-III), lymphadenopathy-associated virus (LAV), and AIDS-associated retrovirus (ARV). Infection with HIV may occur by the virus being transferred by blood, semen, vaginal fluid, or breast milk. Four major means of transmission of HIV include unprotected sexual intercourse, contaminated needles, breast milk, and transmission from an infected mother to her baby at birth.

HIV is an ingeniously constructed very deadly virus, which represents the most challenging pathogen the medical community faces to date. Viruses in general, have been difficult to contain and eradicate due to the fact they are obligate parasites and tend not to carry out any biologic functions outside the cell the virus has targeted as its host. A virus when it exists outside the boundaries of a cell is generally referred to as a virion. HIV virions posses several attributes that make them very elusive and difficult to destroy.

Bacterial infections have posed an easier target for the medical community to eradicate from the body. Bacteria generally live and reproduce outside animal cells. Bacteria, like animal cells, carry out biologic functions. A large multi-celled organism such as the human body combats bacterial infections with a combined force of white cells, antibodies, complements and its lymphatic system. White cells circulate the body in search of bacteria. When a white cell encounters a bacterium, the white cell engulfs the bacterium, encapsulates the pathogen, processes the identification of the pathogen and kills the pathogen utilizing acids and destructive enzymes. The white cell then alerts the B-cells of the immune system as to the identity of the intruding bacterium. A subpopulation of B-cells is generated, dedicated to producing antibodies directed against the particular pathogen the circulating white cell encountered and identified. Antibodies, generated by B-cells, traverse the blood and body tissues in search of the bacteria they were designed to repel. Once an antibody encounters a bacterium it is targeted to attack, the antibody attaches to the bacterium's outer wall. The effect antibodies have in coating the outside of a bacterium is to assist the white cells and the other components of the immune system in recognizing the bacterium, so that appropriate defensive action can be taken against the pathogen. Some antibodies, in addition to coating the bacterium, will act to punch holes through the bacterium's outer wall. If the integrity of the bacterium's cell wall is breached, this action generally leads to the death of the bacterium. Complements are primitive protein structures that circulate the blood stream in search of anything that appears consistent with a bacteria cell wall. Complements are indiscriminant. Once the complement proteins locate any form of bacterial cell wall, the complement proteins organize, and much like antibodies, act in concert to punch one or more holes though a bacterium's cell wall to compromise the viability of the bacterium. As part of the immune system lymphocytes in lymph nodes screen the lymph and cells in the spleen screen the blood in search of bacteria. When a bacterial pathogen is identified, such as by antibodies coating the surface, the bacterium is taken out of circulation and terminated.

Viruses pose a much different infectious vector to the body's defense system than either bacteria or cellular parasites. Since viruses do not carry out biologic processes outside their host cell, a virus can be destroyed, but they cannot be killed. A virus is simply comprised of one or more external shells and a portion of genetic material. The virus's genetic information is carried in the core of the virus. Antibodies can coat the exterior of a virus to make it easier for the white cells in the body to identify the viral pathogen, but the action of punching holes in the virus's external shell by antibodies or complement proteins does not necessarily kill the virus. Viruses also only briefly circulate in the blood and tissues of the body as an exposed entity. Using exterior probes, a virus hunts down a cell in the body that will act as an appropriate host so that the virus can replicate. Once the virus has found a proper host cell, the virus inserts its genome into the host cell. To complete its life-cycle, the virus's genetic material takes command of cellular functions and directs the host cell to make replicas of the virus.

Once the virus's genome has entered a host cell, the virus is in effect shielded from the body's immune system defense mechanisms. Inside a host cell, the presence of the virus is generally only represented as genetic information incorporated into the host cell's DNA. Once a virus has infected a cell in the body, the presence of the virus can only be eradicated if the host cell is destroyed. Antibodies and complements are generally designed not to attack the autologous tissues of the body. Circulating white cells and the immune cells which comprise lymph nodes and the spleen may or may not recognize that a cell, which has become a host for a virus, is infected with a virus's genome. If the immune system fails to identify a cell that has become infected with a virus, the virus's genetic material can proceed to force the infected cell to make copies of the virus. Since a virus is in essence simply a segment of genetic material, time is of no consequence to the life-cycle of the virus and a virus's genome may be carried for years by the host without a need to activate; such viruses are often termed latent viruses. A virus's genetic material may sit idle in a host cell for an extended period of time until the pathogen's programming senses the time is right to initiate the virus's replication process or an action of the host cell triggers the virus to replicate. The only opportunity for the immune system to destroy a latent virus is when copies of the virus leave the host cell and circulate in the blood or tissues in search of another perspective host cell.

The traditional medical approach to combating infectious agents such as bacteria and cellular parasites, therefore has limited value in managing or eradicating elusive or latent viral infections. Synthetic antibiotics, generally used to augment the body's capacity to produce naturally occurring antibodies against bacterial infections, have little success in combating latent viral infections. Stimulating the body's immune system's recognition of a virus by administering a vaccine also has had limited success in combating elusive viral infections. Vaccines generally are intended to introduce to the body pieces of a bacteria or virus, or an attenuated, noninfectious intact bacteria or virus so that the immune system is able to recognize and process the infectious agent and generate antibodies directed to assist in killing the pathogen. Once the immune system has been primed to recognize an intruder, antibodies will be produced by the immune system in great quantities in an effort to repel an invader. Over time, as the immune system down-regulates its antibody production in response to a lack of detecting the presence of the intruding pathogen, the quantity of antibodies circulating in the blood stream may decrease in number to a quantity that is insufficient to combat a pathogen. Since antibodies have limited value in combating some of the more elusive viruses that hibernate in host cells, vaccines have limited value in destroying latent viruses.

The Human Immunodeficiency Virus demonstrates four factors which make this pathogen particularly elusive and a difficult infectious agent to eradicate from the body. First: the host for HIV is the T-Helper cell. The T-Helper cell is a key element in the immune system's response since it helps coordinate the body's defensive actions against pathogens seeking to invade the body's tissues. In cases of a bacterial infection versus a viral infection, T-Helper cells actively direct which immune cells will rev-up in response to the infectious agent and engage the particular pathogen. Since HIV infects and disrupts T-Helper cells, coordination of the immune response against the virus is disrupted, thus limiting the body's capacity to mount a proper response against the presence of the virus and produce a sufficient action to successfully eradicate the virus.

Second: again, latent viruses such as HIV, have a strategic advantage. When the immune system first recognizes a pathogen and begins to generate antibodies against a particular pathogen, the response is generally robust. Once time has passed and the immune system fails to detect an active threat, the production of antibodies against the particular pathogen diminishes. When HIV infects a T-Helper cell, the viral genome may lay dormant, sometimes for years before taking command of the T-Helper cell's biologic functions. HIV may, therefore, generate a very active initial immune response to its presence, but if the virus sits dormant inside T-Helper cells for months or years, the antibody response to the virus will diminish over time. There may not be an adequate quantity of circulating antibodies to actively engage the HIV virions as they migrate from the T-Helper cell that generated the copies to uninfected T-Helper cells that will serve as a new host to support further replication. If the immune system's response is insufficient during the period while the virus is exposed and vulnerable, it becomes extremely difficult for the body to eradicate the virus.

Third, when replicas of the Human Immunodeficiency Virus are released from their host cell, during the budding process, the HIV virion coats itself with an exterior envelope comprised of a portion of the plasma membrane from the T-Helper cell that acted as the host for the virus. A T-Helper cell's plasma membrane is comprised of a lipid bilayer, a double layer of lipid molecules oriented with their polar ends at the outside of the membrane and the nonpolar ends in the membrane interior. The virus thus, in part, takes on an external appearance of a naturally occurring cell in the body. Since the exterior envelope of a HIV virion has the characteristics of a T-Helper cell it is more difficult for the immune system to recognize that it is a pathogen as it migrates through the body in search of another T-Helper cell to infect.

The Human Immunodeficiency Virus posses a fourth, very elusive mode of action, which the virus readily utilizes to actively defeat the body's immune system. HIV carries in its genome a segment of genetic material that directs an infected T-Helper cell to create and mount on the surface the plasma membrane a FasL cell-surface receptor. Healthy T-Helper cells carry on the surface of their plasma membrane Fas cell-surface receptors. The Fas cell-surface receptor when engaged by a FasL cell-surface receptor on another cell, initiates apoptosis in the cell carrying the Fas cell-surface receptor. Apoptosis is a biologic process that causes a cell to terminate itself. A T-Helper cell infected with the HIV virus carrying a FasL cell-surface receptor is therefore capable of killing noninfected T-Helper cells that the infected T-Helper cell encounters as it circulates the body. The occurrence of AIDS is therefore propagated not only by the number of T-Helper cells that become incapacitated due to direct infection by HIV, but also by the number of noninfected T-Helper cells that are eliminated by coming in direct contact with infected T-Helper cells.

Acquired Immune Deficiency Syndrome (AIDS) occurs as a result of the number of circulating T-Helper cells declining to a point where the immune system's capacity to mount a successful response against opportunistic infectious agents is significantly compromised. The number of viable T-Helper cells declines either because they become infected with the HIV virus or because they have been killed by encountering a T-Helper cell infected with HIV. When there is an insufficient population of non-HIV infected T-Helper cells to properly combat infectious agents such as Pneumocystis carinii or cytomegalo virus or other pathogens, the body becomes overwhelmed with the opportunistic infection and the patient becomes clinically ill. In cases where the combination of the patient's compromised immune system and medical assistance in terms of synthetic antibiotics intended to combat the opportunistic pathogens, fluids, intravenous nutrition and other treatments are not sufficient to sustain life, the body succumbs to the opportunistic infection and death ensues.

The Human Immunodeficiency Virus locates its host by utilizing probes located on its envelope. The HIV virion has two types of glycoprotein probes attached to the outer surface of its exterior envelope. A glycoprotein is a structure comprised of a protein component and a lipid component. HIV utilizes a glycoprotein 120 (gp 120) probe to locate a CD4 cell-surface receptor on the plasma membrane of a T-Helper cell. The plasma membrane of the T-Helper cell is comprised of a lipid bilayer. Cell-surface receptors are anchored in the lipid bilayer. Once an HIV gp 120 probe has successfully engaged a CD4 cell-surface receptor on a T-Helper cell a conformational change occurs in the gp 120 probe and a glycoprotein 41 (gp 41) probe is exposed. The gp 41 probe's intent is to engage a CXCR4 or CCR5 cell-surface receptor on the plasma membrane of the same T-Helper cell. Once a gp 41 probe on the HIV virion engages a CXCR4 or CCR5 cell-surface receptor, the HIV virion opens an access portal through the T-Helper cell's plasma membrane.

Once the virus has gained access to the T-Helper cell by opening a portal through the cell's outer membrane the virion inserts two positive strand RNA molecules approximately 9500 nucleotides in length. Inserted along with the RNA strands are the enzymes reverse transcriptase, protease and integrase. Once the virus's genome gains access to the interior of the T-Helper cell, in the cytoplasm the pair of RNA molecules are transformed to deoxyribonucleic acid by the reverse transcriptase enzyme. Following modification of the virus's genome to DNA, the virus's genetic information migrates to the host cell's nucleus. In the nucleus, with the assistance of the integrase protein, the virus's DNA becomes inserted into the T-Helper cell's native DNA. When the timing is appropriate, the now integrated viral DNA, becomes read by the host cell's polymerase molecules and the virus's genetic information commands certain cell functions to carry out the replication process to construct copies of the human deficiency virus.

Present anti-viral therapy has been designed to target the enzymes that assist the HIV genome with the replication process. Anti-viral therapy is intended to interfere with the action of these replication enzymes. Part of the challenge of eradicating HIV is that once the virus inserts its genome into a T-Helper cell host, the viral genome may lay dormant until the proper circumstances evolve. The virus's genome may sit idle inside a T-Helper cell for years before becoming activated, causing drugs that interfere with HIV's life cycle to have limited effect on eliminating the virus from the body. Arresting the replication process does not insure that T-Helper cells infected with HIV do not continue to circulate the body killing noninfected T-Helper cells thus causing the patient to progress to a clinically apparent state of Acquired Immune Deficiency Syndrome and eventually succumbing to an opportunistic infection which eventually results in the death of the individual.

The outer layer of the HIV virion is comprised of a portion of the T-Helper cell's outer cell membrane. In the final stage of the replication process, as a copy of the HIV capsid, carrying the HIV genome, buds through the host cell's plasma membrane, the capsid acquires as its outermost shell a wrapping of lipid bilayer from the host cell's plasma membrane. Vaccines are generally comprised of pieces of a virus or bacterium, or copies of the entire virus or bacterium weakened to the point the pathogen is incapable of causing an infection. These pieces of a pathogen or copies of a nonvirulent pathogen prime the immune system such that a vaccine intent is to cause B-cells to produce antibodies that are programmed to seek out the surface characteristics of the pathogen comprising the vaccine. In the case of HIV, since the surface of the pathogen is an envelope comprised of lipid bilayer taken from the host T-Helper cell's plasma membrane, a vaccine comprised of portions of the exterior envelope of the HIV virions might not only target HIV virions, but might also have deleterious effects on the T-Helper cell population. Some antibodies produced to combat HIV infections may not be able to tell the difference between an HIV virion and a T-Helper cell, and such antibodies may act to coat and assist in the elimination of both targets. In such a scenario, since such a vaccine might cause a decline in the number of available T-Helper cells, it is conceivable that a vaccine comprised of portions of the external envelope of HIV virions might paradoxically induce clinically apparent AIDS in a patient that a vaccine has been administered.

It is clear that the traditional approach of utilizing antibiotics or providing vaccines to stimulate the immune system to produce endogenous antibodies, by themselves, is an ineffective strategy to manage a virus as elusive and deadly as HIV. Drugs that interfere with the replication process of HIV generally slow progression of the infection by the virus, but do not necessarily eliminate the virus from the body nor eliminate the threat of the clinical symptoms of AIDS. A new strategy is required in order to successfully combat the threat of HIV.

Dialysis is generally thought of as a means of removing waste products in patients whose kidneys are no longer capable of effectively filtering the blood and eliminating waste from the body. One option immediately available to reduce the load of HIV virions circulating in the blood would be to physically remove HIV virions from the blood by utilizing a surrogate target to engage HIV. Dialysis utilizes the fact that waste products in the blood are smaller in size than blood cells, therefore by passing blood by a porous filter, blood cells and large proteins can be retained while waste products are separated from the blood cells. Reducing the load of HIV virions circulating in the blood reduces the number of T-Helper cells becoming infected with HIV and forestalls the onset of AIDS.

HIV virions are much smaller in size than red blood cells and white blood cells that circulate in the blood. The blood cells, when removed from the blood, leaves the fluid portion of the blood which is often referred to as plasma. Once the cells have been removed, the fluid portion of the blood could be filtered and HIV separated from the plasma. The gp120 and gp 41 probes located on the surface of HIV are seeking to engage the CD4 and CXCR4 or CCR5 cell-surface receptors located on T-Helper cells. A filter device could be fashioned to be comprised of a chamber of circulating exogenous T-Helper cells coalesced as a collection of T-Helpers previously removed from the patient or a collection of T-Helpers pooled from blood bank donors or a collection of T-Helper cells artificially cultured outside the human body. Blood plasma taken from a patient infected with HIV would be introduced into the filter chamber as simultaneously blood plasma would be removed from the filter chamber. As blood plasma passes through the filter chamber, HIV would come in contact with the collection of exogenous T-Helper cells. As HIV's glycoprotein probes engaged the cell-surface receptors mounted on the exogenous T-Helper cells present in the chamber, the HIV virions would adhere to the exogenous T-Helper cells and either become stuck to the T-Helper cells thus being retained in the filter chamber as the blood plasma exited the chamber, or by the action of the HIV probes engaging the T-Helper cell's cell-surface receptors HIV would eject its genome thus making it incapable of infecting an endogenous T-Helper cell in the patient. The patient's blood plasma, now cleared of infectious HIV virions, could be infused back into the patient.

The technology to make such filtering mechanisms is readably available and could be quickly implemented for worldwide use to treat patients infected with HIV.

A quantity of T-Helper cells has a limited life-span and requires special handling measures to insure the T-Helper cells do not become metabolically inactive and then deteriorate to a point where they are ineffective as a filtering mechanism. A method to accomplish the same task without having to incorporate healthy T-Helper cells would be construct a filter mechanism that houses a material comprised only of the surface materials of a naturally occurring T-Helper cell, since, specifically, it is the cell-surface receptors that HIV virions' probes are seeking. The surface or outer membrane, often referred to as the plasma membrane, of a T-Helper cell is a lipid bilayer. Sheets or strips or spheres of lipid bilayer constructed with a large quantity of CD4, CXCR4 and CCR5 cell-surface receptors affixed to the surface, could be utilized, in place of T-Helper cells, as a surrogate target to attract and engage HIV virions. Since such sheets or strips or spheres of lipid bilayer are not necessarily metabolically active, the storage time may be significantly lengthened in comparison to metabolically active T-Helper cells. Similar to the design of a cell, a sphere comprised of lipid bilayer surface, attached to this surface a large quantity of CD4, CXCR4 and CCR5 cell-surface receptors, could be used as a surrogate target for HIV virions. A sphere comprised of a lipid bilayer shell or surface, with cell-surface receptors attached to the outer surface could potentially be stored and retain their viability for a much longer period of time than metabolically active T-Helper cells.

A filter device comprised of a filter chamber could be constructed in a manner where one or more sheets of lipid bilayer, or one or more strips of lipid bilayer, or a quantity of lipid bilayer spheres, each form of lipid bilayer constructed with a large quantity of CD4, CXCR4 and CCR5 cell-surface receptors, would be placed inside a filter chamber. Blood or blood plasma could be caused to pass through the filter chamber. As the blood or blood plasma passes across the surface of a sheet of lipid bilayer or strip of lipid bilayer or a sphere comprised of lipid bilayer material HIV virions would come in contact with CD4, CXCR4 and CXR5 cell-surface receptors present on the surface of the lipid bilayer material and engage the cell-surface receptors. The HIV virions making contact with the lipid bilayer material would either permanently adhere to the lipid bilayer material or by engaging the cell-surface receptors on the lipid bilayer material the HIV virions would be caused to eject their genome, which would neutralize the infectious threat of the HIV virions. The blood or blood plasma exiting the filter chamber would be cleared of HIV virions capable of infecting a T-Helper cell. This blood or blood plasma would then be reintroduced back into a body.

Since HIV virions are searching their environment for CD4, CXCR4 and CXR5 cell-surface receptors a filter material comprised of any hypoallergenic material with CD4, CXCR4 and CXR5 cell-surface receptors or the protein portion of these receptors attached to the surface of the material could be placed inside the filtering chamber and act as an effective filter medium. Blood or blood plasma could be caused to pass through the filter chamber. As the blood or blood plasma passes across the surface of the hypoallergenic filter medium, HIV virions would come in contact with CD4, CXCR4 and CXR5 cell-surface receptors present on the surface of the hypoallergenic medium and engage the cell-surface receptors. The HIV virions making contact with the cell-surface receptors would either permanently adhere to the hypoallergenic filter medium or by engaging the cell-surface receptors on the hypoallergenic filter medium the HIV virions would be caused to eject their genome, which would neutralize the infectious threat of the HIV virions. The blood or blood plasma exiting the filter chamber would be cleared of HIV virions capable of infecting a T-Helper cell. This blood or blood plasma would then be reintroduced back into a body.

White blood cells are physically larger than red blood cells. Bacteria are generally much smaller than red blood cells. HIV virions are much smaller than bacteria. HIV is comprised of an outer envelope, an internal capsid and the viral genome. Because of its small size HIV can potentially maneuver into places in the tissues where mobile cells are unable to go.

An approach to managing HIV would be to create a product that would be relatively the same size as HIV so that the product could penetrate into every location that HIV might migrate. HIV's probes are seeking the CD4 and CCR5 and CXCR4 cell-surface receptors of a T-Helper cell, thus a product to challenge HIV could be equipped with the same cell-surface receptors as would be found on a naturally occurring T-Helper cell.

Utilizing genetic machinery and a colony of T-Helper cells or a colony of hybrid T-Helper cells or a colony of host cells, a product approximately the size of a HIV virion could be manufactured in a similar manner as how HIV naturally replicates, except the product would carry T-Helper cell cell-surface receptors CD4, CXCR4 and/or CCR5 instead of the glycoprotein probes associated with a naturally occurring HIV virion. The product would be constructed either with no genetic information present inside the capsid or genetic material to act as a filler substance, this genetic material being inert such that it could not carry out any useful function except that of acting as a filler. Such a filler material would help the structure retain a spherical shape.

Constructing a virus-like structure, with the surface characteristics of a virus, that has affixed to its exterior cell-surface receptors intended to engage a virus, is referred to as a Scientifically Modulated And Reprogrammed Target (SMART) virus. Such a structure could be simply a sphere of lipid bilayer material will cell-surface receptors attached to the outer surface as described previously, or such structures may carry a filler substance in order to maintain and retain the integrity of the shape of the structure. Spheres comprised of lipid bilayer material may require a filler substance to retain their spherical shape if the size of the structure becomes very large. Copies of such a SMART virus could be placed in a filter chamber. The diameter of the SMART virus could be increased to a size larger than the naturally occurring HIV virion to facilitate containing the SMART virus inside the filter chamber as the blood or blood plasma passes through the filter chamber. Blood or blood plasma could be passed through the filter chamber containing a quantity of SMART virus. The SMART virus would be available within the walls of the chamber to engage HIV virions as the blood or blood plasma passed through the filter chamber. As HIV virions made contact with SMART viruses the HIV virions would engage the SMART viruses and become permanently attached and become trapped inside the chamber, or a HIV virion, upon engaging a SMART virus, would harmlessly eject the genetic material the HIV virion carries. Either trapping the HIV virion inside the filter chamber or causing the HIV virion to eject the genetic material that it carries, would neutralize the virulence of HIV and assist in managing the threat of AIDS.

### BRIEF SUMMARY OF THE INVENTION

Initially the Human Immunodeficiency Virus is attracted to its host, the T-Helper cell, by having its surface probes seek out a CD4 cell-surface receptor. Once a HIV virion's gp 120 probe successfully engages a CD4 cell-surface receptor a conformation change occurs in the gp 120 probe and a gp 41 probe attempts to engage either a CXCR4 or a CCR5 cell-surface receptor located on the target T-Helper cell. Described here is a device that simulates the target the HIV virions are seeking. It is a device intended to remove infectious Human Immunodeficiency Virus virions from a fluid such as blood or blood plasma. Blood is removed from a patient and this blood enters a filter chamber that contains a filter medium. As the blood transits through the filter chamber the blood makes contact with the filter medium present in the filter chamber. As the blood transits the filter chamber any HIV virions present in the blood have the opportunity to engage the three cell-surface receptors including the CD4 cell-surface receptor, the CCR5 cell-surface receptor and the CXCR4 cell-surface receptor which are well known to the medical and scientific community due to the fact they appear naturally on the surface of the Human T-Helper cell. Since the HIV virion engaged cell-surface receptors located on the surface of the filter medium rather than located on the surface of an endogenous T-Helper cell inside the body, the infectious nature of the HIV virions is neutralized by either the HIV virion becoming trapped inside the filter chamber by being attached the filter medium or the HIV virion is caused to harmlessly eject its genome. When HIV virions become trapped inside the filter chamber they are incapable of successfully engaging endogenous T-Helper cells inside the body. When a HIV virion is caused to eject its genome, the HIV virion is incapable of infecting T-Helper cell inside the body with its genome an endogenous. Trapping the HIV virion or causing the HIV virion to harmlessly eject its genome leads to neutralizing the infectious threat of HIV, which leads to effectively averting AIDS.

### DETAILED DESCRIPTION OF THE INVENTION

The invention described herein is intended to filter infectious Human Immunodeficiency Virus virions from a fluid such as blood or blood plasma. The filtering process may be dynamic such as blood is actively removed from an individual, the blood transits through one or more filtering devices and the cleansed blood is then returned to the same individual. The filtering process may be more static in how it is conducted, where a specific quantity of blood is removed from one individual, the blood products are filtered through one or more filtering devices and this blood or separate blood products now cleansed of infectious HIV virions are, at a later time, infused into one or more individuals in need of such blood products.

Three cell-surface receptors CD4, CCR5 and CXCR4 are well known to the medical and scientific community and appear naturally on the surface of the Human T-Helper cells. The HIV virion expresses glycoprotein 120 (gp 120) probes and glycoprotein 41 (gp 41) probes on its outer envelope. HIV utilizes the T-Helper cell as its host cell for the purposes of replication.

In completing the virus's natural reproductive-cycle, HIV utilizes gp 120 probes positioned on the exterior envelope of a HIV virion to locate and engage a T-Helper cell's CD4 exterior cell-surface receptor. Once a HIV's gp 120 probe has successfully engaged a CD4 cell-surface receptor, a HIV virion's gp 41 probe engages either a CCR5 or CXCR4 exterior cell-surface receptor located on the T-Helper cell. A filter medium present inside the chamber of a filter device, expressing CD4, CCR5 and CXCR4 cell-surface receptors offers the target cell-surface receptors the HIV virions are seeking to engage. When a HIV virion's probes encounter a filter medium expressing CD4, CCR5 and CXCR4 cell-surface receptors, HIV's gp 120 probes would engage CD4 exterior surface receptors, then a HIV's gp 41 probe will engage either a CCR5 or CXCR4 exterior cell-surface receptor. Once the HIV gp 120 and gp 41 probes have engaged their respective receptors on the filter medium's exterior surface, the HIV is fixed to the surface of the filter medium and the HIV virion may eject its RNA genome payload. Since the HIV engaged a filter medium inside the filtering device the HIV virion becomes trapped inside the filter device and if the HIV virion ejects its RNA genome, the threat of the HIV virion being able to infect an endogenous T-Helper cell inside a body is effectively neutralized. The fluid, such as blood plasma, passing through such a filter becomes cleared of infectious HIV virions.

The medical device described herein, intended to remove infectious HIV virions from blood plasma, is comprised of a chamber, where blood plasma is introduced into the chamber at one location, the blood plasma comes into contact with a filter medium, the blood plasma exits the chamber at a different location than where the blood plasma entered the chamber. The filter medium inside the filter chamber may be comprised of several different materials and designs. The filter medium is intended to make available cell-surface receptors including CD4, CCR5 and CXCR4 for HIV virions to engage. The filter medium may be comprised of a quantity of exogenous T-Helper cells. The filter medium may be comprised of a quantity of lipid bilayer sheets which are comprised of similar materials as found existing as the outer membrane of a T-Helper cell, and affixed to the said lipid bilayer sheets are glycoprotein cell-surface receptors including a quantity of CD4 cell-surface receptors, CXCR4 cell-surface receptors, CCR5 cell-surface receptors. Such bilayer sheets may be of any suitable shape which might include such shapes as the shape of a square, the shape of a rectangle, the sheet may be attached to itself to be the shape of a cylinder. The filter medium may be comprised of a quantity of lipid bilayer strips which are comprised of similar materials as found existing as the outer membrane of a T-Helper cell, and affixed to the said lipid bilayer strips are cell-surface receptors including a quantity of CD4 cell-surface receptors, CXCR4 cell-surface receptors, CCR5 cell-surface receptors. Such strips may be long and thin with the dimension of the length greater than the dimension of the width, and may include any suitable shape such as a long thin strand or the shape of a coil or one end may be attached to another end to form the shape of a ring or circle. The filter medium may be comprised of a quantity of lipid bilayer spheres which are comprised of similar materials as found existing as the outer membrane of a T-Helper cell, and affixed to the said lipid bilayer spheres are cell-surface receptors including a quantity of CD4 cell-surface receptors, CXCR4 cell-surface receptors, CCR5 cell-surface receptors. The shapes of the spheres may include any suitable shape such as the shape of a ball, the shape of cylinder, the shape of an ellipsoid. The filter medium may be comprised of a quantity of modified viruses or virus-like structures with cell-surface receptors to include a quantity of CD4 cell-surface receptors, CXCR4 cell-surface receptors, CCR5 cell-surface receptors. The filter medium may be comprised of any suitable hypoallergenic material, which can be affixed to the surface a quantity of CD4 cell-surface receptors, CXCR4 cell-surface receptors, CCR5 cell-surface receptors or simply the protein portion of the CD4 cell-surface receptors, CXCR4 cell-surface receptors, CCR5 cell-surface receptors. The shape of the hypoallergenic material may include a variety of suitable shapes including the shape of a sheet, shape of a strip or shape of a sphere.

The material to be used to create the walls of such a filter chamber may include any suitable material such as glass, rigid plastic, a flexible plastic, latex, steel, aluminum or other metal or metal alloy. A tube to carry blood or blood plasma to the filter chamber would be attached to the portal where the blood or blood plasma would enter the filter chamber. A tube would be attached to the portal of the filter chamber where the blood or blood plasma would exit the chamber to carry the filtered blood or blood plasma away from the chamber. The tubing carrying blood or blood plasma to the filter chamber and the tubing carrying blood or blood plasma away from the filter chamber would be comprised of materials such as a flexible plastic, rigid plastic, a flexible metal or a rigid metal or latex. A porous barrier located at the portal where the blood or blood plasma enters the filter chamber and a porous barrier located at the portal where the blood or blood plasma exits the filter chamber would be comprised of materials such as a flexible plastic, a rigid plastic, a flexible metal or a rigid metal or latex. The said porous barriers are comprised of a quantity of holes, said holes large enough to allow said blood or blood plasma to freely enter and exit said chamber, but said holes are restrictive enough so as to retain said filter medium inside the inner boundaries of said chamber as said blood or blood plasma transits through said chamber.

To carry out the process to manufacture a modified medically therapeutic virus or virus-like structure, DNA or RNA code that would provide the necessary biologic instructions to generate the general physical outer structures of the modified virus or virus-like structure, would be inserted into a host. The host may include devices such as a host cell or a hybrid host cell. The host may utilize DNA or RNA or a combination of genetic instructions in order to accomplish the construction of medically therapeutic modified virus virions or virus-like structures. In some cases DNA or messenger RNA would be inserted into the host that would be coded to cause the production of cell-surface receptors that would be affixed to the surface of the modified virus virion or virus-like structure that would target the glycoprotein probes affixed to the surface of an HIV virion. The copies of the medically therapeutic modified viruses or medically therapeutic virus-like structures, upon exiting the host, would be collected, stored and utilized as a filter medium in the described filter chamber as necessary.

The medically therapeutic version of the modified virus and virus-like structures would be incapable of replication on its own due to the fact that the messenger RNA that would code for the replication process to produce copies of the virus or virus-like structure would not be present in the modified form of a virus or virus-like structure.

Lipid bilayer sheets, strips, spheres can be manufactured and combinations of CD4 cell-surface receptors, CXCR4 cell-surface receptors, and CCR5 cell-surface receptors can be affixed to the surface with the structure acting as a filter medium. Sheets of any suitable hypoallergenic material can be manufactured and combinations of CD4 cell-surface receptors, CXCR4 cell-surface receptors, and CCR5 cell-surface receptors can be affixed to the surface with the structure acting as a filter medium. Sheets of any suitable hypoallergenic material can be manufactured and combinations of the protein portion of the CD4 cell-surface receptors, CXCR4 cell-surface receptors, and CCR5 cell-surface receptors attached to the surface of the hypoallergenic surface and made available to engage the glycoprotein probes affixed to the surface of HIV virions with the structure acting as a filter medium.

The invention described herein is intended to filter infectious Human Immunodeficiency Virus virions from a fluid such as blood or blood plasma. The filtering process may be dynamic such as blood that is actively removed from an individual, the blood transits through one or more filtering devices and the cleansed blood is then returned to the same individual. In the filtering process as the blood from the individual makes contact with the filter medium inside the filter chamber to filter out or neutralize HIV virions present in blood or blood plasma. Blood cleansed of infectious HIV virions is returned to the same individual.

The filter device may be used in a more static process, where a specific quantity of blood is removed from one individual, the blood products transit through one or more filtering devices and this now cleansed blood or separate blood products are, at a later time, infused into one or more other individuals in need of such cleansed blood products. The blood permanently removed from the first individual makes contact with the filter medium inside the filter chamber which filters out or neutralizes HIV virions present in blood or blood plasma. Blood removed from the first individual, now cleansed of infectious HIV virions, is then provided to one or more other individuals requiring such blood products.

### DRAWINGS

None.

What is claimed:

1. A medical device to remove virus virions from blood comprised of a chamber:
(a) where blood enters said chamber at one location,
(b) said blood comes into contact with a filter medium,
(c) said filter medium having cell-surface receptors affixed to its surface,
(d) said blood exits said chamber at a different location than where said blood entered said chamber,
(e) said filter medium is retained inside said chamber,
whereby virus virions are intended to come in contact with said cell-surface receptors found on the surface of said filter medium contained inside said chamber,
whereby virus virions are intended to engage said cell-surface receptors found on the surface of said filter medium contained inside said chamber with the intention of preventing said virus virions from being able to infect host cells endogenous to a body by trapping the virus virions inside said filter chamber or by neutralizing the infectious threat posed by said virus virions by causing said virus virions to harmlessly eject the genetic genome said virus virions carry.

2. The medical device in claim 1 wherein said blood represents the fluid portion of blood, which does not include the cellular structures of white blood cells and red blood cells found in whole blood.

3. The medical device in claim 1 wherein said blood is removed from said body, said blood transits the medical device and said blood is returned to said body.

4. The medical device in claim 1 wherein at about the location of said portal where said blood enters said chamber, a porous barrier is present comprised of a quantity of holes, said holes sufficient in the size of their dimensions to allow said blood to freely enter said chamber, but said holes are restrictive enough in the size of their dimensions so as to retain said filter medium inside the inner boundaries of said chamber as said blood transits through said chamber.

5. The medical device in claim 1 wherein at about the location of said portal where said blood exits said chamber, a porous barrier is present comprised of a quantity of holes, said holes sufficient in the size of their dimensions to allow said blood to freely exit said chamber, but said holes are restrictive enough in the size of their dimensions so as to retain said filter medium inside the inner boundaries of said chamber as said blood transits through said chamber.

6. The medical device in claim 1 wherein said filter medium selected from the group consisting of a quantity of T-Helper cells, a quantity of lipid bilayer material in the shape of a sheet, a quantity of lipid bilayer material in the shape of a strip, a quantity of lipid bilayer material in the shape of a sphere, a quantity of modified virus virions, and a quantity of virus-like structures.

7. The medical device in claim 1 wherein said filter medium selected from the group consisting of a quantity of hypoallergenic surfaces in the shape of a sheet capable of acting as a base on which can be affixed said cell-surface receptors to support the functional expression of a quantity of said cell-surface receptors, a quantity of hypoallergenic surfaces in the shape of a strip capable of acting as a base on which can be affixed said cell-surface receptors to support the functional expression of a quantity of said cell-surface receptors, and a quantity of hypoallergenic surfaces in the shape of a sphere capable of acting as a base on which can be affixed said cell-surface receptors to support the functional expression of a quantity of said cell-surface receptors.

8. A medical device to remove Human Immunodeficiency Virus virions from blood comprised of a chamber:
(a) where blood enters said chamber through a portal at one location,
(b) said blood comes into contact with a filter medium,
(c) said filter medium having cell-surface receptors affixed to its surface,
(d) said blood exits said chamber through a different portal at a different location than where said blood entered said chamber,
(e) said filter medium is retained inside said chamber,
whereby Human Immunodeficiency Virus virions are intended to come in contact with said cell-surface receptors found on the surface of said filter medium contained inside said chamber,
whereby said Human Immunodeficiency Virus virions are intended to engage said cell-surface receptors found on the surface of said filter medium contained inside said chamber with the intention of preventing said Human Immunodeficiency Virus virions from being able to infect T-Helper cells endogenous to a body by trapping said Human Immunodeficiency Virus virions inside said filter chamber or by neutralizing the infectious threat posed by said Human Immunodeficiency Virus virions by causing said Human Immunodeficiency Virus virions to harmlessly eject the genetic genome said Human Immunodeficiency Virus virions carry.

9. The medical device in claim 8 wherein said blood represents the fluid portion of blood, which does not include the cellular structures of white blood cells and red blood cells found in whole blood.

10. The medical device in claim 8 wherein said blood is blood removed from said body, said blood transits the medical device and said blood returns to said body.

11. The medical device in claim 8 wherein at about the location of the portal where said blood enters said chamber a porous barrier is present comprised of a quantity of holes, said holes are sufficient in the size of their dimensions to allow said blood to freely enter the chamber, but said holes are restrictive enough in the size of their dimensions so as to retain said filter medium inside the inner boundaries of said chamber as said blood transits through said chamber.

12. The medical device in claim 8 wherein at about the location of the portal where said blood exits said chamber a porous barrier is present comprised of a quantity of holes, said holes are sufficient in the size of their dimensions to allow said blood to freely exit said chamber, but said holes are restrictive enough in the size of their dimensions so as to retain said filter medium inside the inner boundaries of said chamber as said blood transits through said chamber.

13. The medical device in claim 8 wherein said chamber is in the shape of a tube where said chamber is fashioned to wrap around onto itself such that one end is affixed to the other end such that said filter medium is present inside said chamber and said filter medium is retained inside said chamber as said blood enters the chamber through said entry portal, transits said chamber while doing so said blood makes contact with the surface of said filter medium, then said blood exits said chamber through said exit portal in the filter chamber.

14. The medical device in claim 8 wherein an initiative to move said blood through said chamber is executed by roller devices that apply gentle compressive and decompressive forces on the exterior of said chamber to create a flow of said blood through said chamber from said entry portal to said exit portal of said chamber.

15. The medical device in claim 8 wherein said filter medium is a quantity of T-Helper cells.

16. The medical device in claim 8 wherein said filter medium is selected from the group consisting of a quantity of modified virus virions and a quantity of virus-like structures.

17. The medical device in claim 8 wherein said filter medium selected from the group consisting of a quantity of lipid bilayer sheets, a quantity of lipid bilayer strips, and a quantity of lipid bilayer spheres.

18. The medical device in claim 8 wherein said filter medium selected from the group consisting of a quantity of hypoallergenic surfaces in the shape of a sheet capable of acting as a base on which can be affixed said cell-surface receptors to support the functional expression of a quantity of said cell-surface receptors, a quantity of hypoallergenic surfaces in the shape of a strip capable of acting as a base on which can be affixed said cell-surface receptors to support the functional expression of a quantity of said cell-surface receptors, and a quantity of hypoallergenic surfaces in the shape of a sphere capable of acting as a base on which can be affixed said cell-surface receptors to support the functional expression of a quantity of said cell-surface receptors.

19. The medical device in claim 8 wherein said cell-surface receptors selected from the group consisting of a quantity of CD4 cell-surface receptors, a quantity of CXCR4 cell-surface receptors and a quantity of CCR5 cell-surface receptors.

20. The medical device in claim 8 wherein said cell-surface receptors are comprised of a quantity of CD4 cell-surface receptors, a quantity of CXCR4 cell-surface receptors and a quantity of CCR5 cell-surface receptors.
